(19) United States
(12) Patent Application Publication
(10) Pub. No.: US 2007/0208204 A1
(43) Pub. Date: Sep. 6, 2007

(54) POLYMERIZATION INHIBITOR FOR STABILIZING OLEFINICALLY UNSATURATED MONOMERS

(75) Inventors: Oliver Meyer, Munster (DE); Phillip James, Romsey/Hampshire (GB); Oliver Erpeldinger, Wulfrath (DE); Frank Kraushaar, Essen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA GmbH, Duesseldorf (DE)

(21) Appl. No.: 11/671,119

(22) Filed: Feb. 5, 2007

(30) Foreign Application Priority Data
Mar. 3, 2006 (DE) 10 2006 010 347.5

(51) Int. Cl. C10L 1/06; C07C 7/20

(52) U.S. Cl. 585/4; 585/1; 585/2

(57) ABSTRACT

A polymerization inhibitor for stabilizing olefinically unsaturated monomers containing at least one compound of formula (1) and/or (2) according to the invention. Stabilized monomer compositions.

### REFERENCE TO RELATED CASES

This application is based on German Patent Application No. 102006010347.5 filed Mar. 3, 2006, the contents of which are expressly incorporated by reference herein.

### BACKGROUND OF THE INVENTION

1. Field of the Invention

The invention relates to the use of a polymerization inhibitor for stabilizing olefinically unsaturated monomers, and to a monomer composition comprising both olefinically unsaturated monomers and the polymerization inhibitor.

During the preparation of olefinically unsaturated monomers, for example, ethene, butadiene, vinyl acetate, (meth)acrylic acid, (meth)acrylate, acrylonitrile or styrene, these olefinically unsaturated monomers are subjected to a purification process step, for example, distillation or extraction, in order to remove undesired by-products or impurities.

These olefinically unsaturated monomers can polymerize as early as during the preparation and/or purification process. Some of these olefinically unsaturated monomers, for example, butadiene, tend to polymerize spontaneously even while being stored or transported.

This premature and undesired polymerization of these olefinically unsaturated monomers leads firstly to a reduction in the amount of usable olefinically unsaturated monomers, and secondly to undesired deposition of undesired polymer. This deposition of the undesired polymer can lead under some circumstances to reduced heat transfer in individual plant parts. Moreover, surfaces that are coated with the undesired polymer, or plant parts, for example, filters, that are blocked by the undesired polymer, can lead to interruption of production.

Consequently, additives are generally added to the olefinically unsaturated monomers as early as in the preparation process, which are referred to either as polymerization inhibitors or as polymerization retardants, which are capable either of preventing the undesired polymerization process or at least of retarding it.

2. Discussion of Prior Art

A multitude of polymerization inhibitors is known for olefinically unsaturated monomers, for example, styrene. Examples of compounds used for the purpose include sulfur, p-benzoquinone, 4-tert-butylpyrocatechol, phenothiazine or sterically hindered phenols. However, some of these compounds have considerable disadvantages, for example, their toxicity, instability at relatively high temperatures or insufficient activity under the appropriate process conditions of the preparation or purification process. Some of the polymerization inhibitors mentioned even require oxygen to be able to display their action, which can lead to considerable problems with regard to the explosion protection for use in industrial scale processes.

Polymerization inhibitors frequently described in the literature are so-called stable free nitroxyl radicals, for example, 2,2,6,6-tetramethylpiperidine N-oxyl (frequently abbreviated as TEMPO). U.S. Pat. No. 3,747,988 describes the addition of a nitroxyl radical, especially of 2,2,6,6-tetramethyl-4-hydroxy-piperidine N-oxyl, to acrylonitrile before distillation. These stable free radicals are also described in U.S. Pat. No. 3,488,338, except as chain terminators for 2-chloro-1,3-butadiene. U.S. Pat. No. 4,670,131 describes the use of these stable free nitroxyl radicals as a polymerization inhibitor for stabilizing olefinically unsaturated monomers, for example, ethene, propene, butene or butadiene.

The use of stable free nitroxyl radicals is described, inter alia, by the following publications “Inhibition of Radical Polymerization by Nitroxide Mono- and Biradicals” (Vysokomol, Soyed. 8: 1966, No. 9, 1642-1646) by L. V. Ruban et al., “A Re-Examination of Some Stabilized Radicals as Inhibitors of Polymerization” (J. Polym. Mater. 19 (2002), 113-120) and “Stabilized Radicals as Inhibitors of Polymerization—Reactions of Alkoxyamines with Growing Polymer Radicals” (J. Macrom. Science 2002, Vol. A39, No. 11, 1295-1303) by Bevington et al.

Numerous publications describe the use of compositions comprising, inter alia, stable free nitroxyl radicals. For instance, U.S. Pat. No. 6,525,146, WO 2002/088055 and EP 1 077 245 describe a composition composed of stable free nitroxyl radicals and phenol derivatives as a polymerization inhibitor. Further examples of compositions for the stabilization of olefinically unsaturated monomers that have stable free nitroxyl radicals are described in US 2003/080318, WO 2002/094884, WO 2002/033026, WO 2002/00816, EP 1 077 206 and DE 199 56 509.

### OBJECT OF THE INVENTION

It is therefore an object of the present invention to provide a polymerization inhibitor for olefinically unsaturated monomers with improved action over the prior art. In particular, a polymerization inhibitor is provided that has improved properties in the stabilization of olefinically unsaturated monomers with respect to premature polymerization during the preparation and purification process or during storage.

### DESCRIPTION OF THE INVENTION

It has been found that, surprisingly, compounds of the formula (1) and/or (2) are suitable as polymerization inhibitors for olefinically unsaturated monomers. The preparation of these compounds has been known for some time, but it has not been recognized that these compounds can be used as polymerization inhibitors. It was also surprising that these compounds which do not possess a stable free radical can display this action as a polymerization inhibitor. The solution to this problem was all the more surprising given that it was found that the action of these compounds as polymerization inhibitors is improved over prior art polymerization inhibitors that have a stable free radical in their structure. With the same molar amount of polymerization inhibitor in the olefinically unsaturated monomers, a lower polymer content in the olefinically unsaturated monomers can be obtained by the inventive use compared to the prior art. Alternatively, the same polymer fraction in the olefinically unsaturated monomers can be achieved by a smaller molar amount of polymerization inhibitor.

The invention provides for the use of a polymerization inhibitor for stabilizing olefinically unsaturated monomers, the polymerization inhibitor comprising, among other possible ingredients,

from 0 to 100% by weight of a compound of the formula (1)

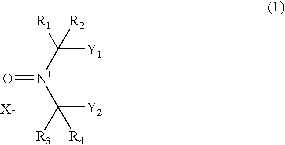

and

from 100 to 0% by weight of a compound of the formula (2)

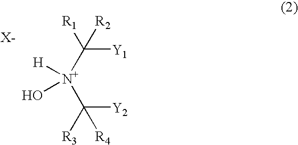

wherein
R₁, R₂, R₃ and R₄=alkyl group having from 1 to 4 carbon atoms,
Y₁, Y₂=alkyl group having from 1 to 4 carbon atoms, or Y₁ and Y₂ together form a ring system, and
X⁻=inorganic or organic anion,
   where the substituents of the R₁, R₂, R₃, R₄, Y₁ and Y₂ type are the same or different, the substituents Y₁ and Y₂ and the ring system based on the substituents Y₁ and Y₂ may be unsubstituted or substituted, where the sum of the percentages by weight of the compounds of the formula (1) and (2) adds up to 100% by weight based on the weights of compounds of those formulae, and being mixed with at least one olefinically unsaturated monomer or a monomer mixture comprising at least one olefinically unsaturated monomer. Said another way, the invention polymerization inhibitor comprises at least one compound of formulae (1) and/or (2), and during use is mixed with one or more olefinically unsaturated monomers or a monomer mixture comprising at least one olefinically unsaturated monomer.

The invention further provides a monomer composition, the monomer composition comprising a polymerization inhibitor and at least one olefinically unsaturated monomer, the polymerization inhibitor comprising at least one compound of formulae (1) and (2).

Preferred inventive compounds of formulae (1) and (2) are depicted below:

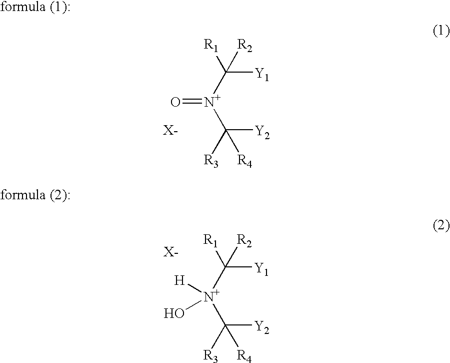

wherein

R₁, R₂, R₃ and R₄=alkyl group having from 1, 2, 3 or 4 carbon atoms,

Y₁, Y₂=alkyl group having from 1, 2, 3 or 4 carbon atoms, or Y₁ and Y₂ together form a ring system, and

X⁻=inorganic or organic anion,

where the substituents of the R₁, R₂, R₃, R₄, Y₁ and Y₂ type are the same or different, either the substituents of the Y₁ and Y₂ type or the ring system based on the substituents of the Y₁ and Y₂ type may be unsubstituted or substituted.

In the inventive use of these polymerization inhibitors, compounds of formula (1) and/or of formula (2) may be used. However, preference is given to using a composition as the polymerization inhibitor comprising both compounds of formula (1) and of formula (2). Multiple compounds of each formula may be used, if desired. Preferably, a polymerization inhibitor is used comprising, relative to the amounts of formulae (1) and (2) therein:

from 40 to 60% by weight of a compound of the formula (1) and

from 60 to 40% by weight of a compound of the formula (2).

It is particularly preferred to use a polymerization inhibitor comprising

from 45 to 55% by weight of a compound of the formula (1) and

from 55 to 45% by weight of a compound of the formula (2).

The polymerization inhibitors used in the invention can be prepared in a process as described by Merbouh et al. in “Preparation of tetramethylpiperidine-1-oxoammonium salts and their use as oxidants in organic chemistry. A review” (Organic Preparations and Procedures International, 2004, 36(1), 3-31) or Golubev et al. in “Some reactions of free iminoxyl radicals with the participation of the unpaired electron” (Seriya Khimicheskaya, No. 11, 1965, 1927-1936).

In a preferred process for preparing these compounds of the formula (1), generally both compounds of the formula (1) and of the formula (2) are formed; in this process, the appropriate nitroxyl radical is reacted in solution with a strong inorganic or organic acid. The disproportionation of the nitroxyl radical forms an approx. 1:1 mixture of the corresponding oxoammonium salt of the formula (1) and of the corresponding hydroxylamine compound of the formula (2). Therefore, when a composition of these compounds of the formula (1) and (2) is used according to the invention, a complicated separation of the two compounds after the preparation process can be dispensed with.

In a particular embodiment of these polymerization inhibitors according to the invention, they are prepared in situ.

In a particular embodiment of the invention, the substituents of the Y₁ and Y₂ type are substituted, the substituent or these substituents of the substituents of the Y₁ and Y₂ type being selected from alkyl, ester, ether, hydroxyl, oxo, cyano, cyanohydrin, amino, amide, carboxyl, halogen, hydantoin, ketal, acetal or urethane group.

The two substituents of the Y₁ and Y₂ type of the polymerization inhibitor together preferably form a ring system. In the invention, particular preference is given to using a polymerization inhibitor which comprises at least one compound according to formula (3) and/or (4):

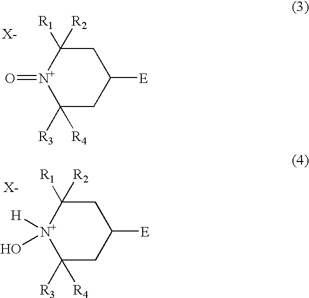

wherein

R₁, R₂, R₃ and R₄=alkyl group having from 1 to 4 carbon atoms,

E=alkyl, ester, ether, hydroxyl, oxo, cyano, cyanohydrin, amino, amide, carboxyl, halogen, hydantoin, ketal, acetal or urethane group and

X⁻=inorganic or organic anion,

where the substituents of the R₁, R₂, R₃ and R₄ type are the same or different. In the invention, very particular preference is given to using a polymerization inhibitor comprising compounds of the formula (3) and/or (4) where R₁, R₂, R₃ and R₄=methyl group.

In particular, in the invention, polymerization inhibitors are used that have an inorganic or an organic anion in the compounds of the formulae (1) to (4); the polymerization inhibitors used preferably have inorganic anions. In the invention, particular preference is given to using polymerization inhibitors that have, as the anion X— in the compounds of the formulae (1) to (4), a halogen anion, preferably a chlorine or a bromine anion.

In the context of this invention, a polymerization inhibitor is understood to mean a compound that is capable of preventing polymerization of the olefinically unsaturated monomer for a certain period. The period until polymerization occurs in the case of an olefinically unsaturated monomer without a polymerization inhibitor is therefore shorter than the period for an olefinically unsaturated monomer with a polymerization inhibitor.

Moreover, in the context of this invention, olefinically unsaturated monomers are understood to mean compounds that have at least one C—C double bond and are capable of entering into a polymerization reaction.

In the invention, preference is given to using at least one olefinically unsaturated monomer selected from alk-1-enes or alka-1,3-dienes which may be either unsubstituted or substituted. Preference is given to using olefinically unsaturated monomers selected from ethene, propene or propylene, butadiene, vinyl acetate, (meth)acrylic acid, (meth)acrylate, acrylonitrile, acrolein, N-vinylformamide, chloroprene, isoprene, divinylbenzene or styrene.

In the context of this invention, (meth)acrylic acid means both acrylic acid and methacrylic acid, and (meth)acrylate means both acrylic esters and methacrylic esters, these olefinically unsaturated monomers being substituted or unsubstituted.

In the invention, it is possible to use either one compound of olefinically unsaturated monomer or a mixture of different olefinically unsaturated monomers.

In the invention, the polymerization inhibitors may be added to the olefinically unsaturated monomers as a solid, solution or as a suspension. In this case, the polymerization inhibitors may also be added to the olefinically unsaturated monomers during a process, for example, a preparation or purification process. It is advantageous, in the use of these polymerization inhibitors according to the invention, to ensure a suitable solvent or dispersant that is compatible firstly with the olefinically unsaturated monomer and also with these polymerization inhibitors, and that there are no undesired reactions.

These polymerization inhibitors can be added to the unsaturated monomers or monomer mixtures using common prior art methods. Advantageously, in the invention, the polymerization inhibitors can be added to the feed stream of a distillation column, into the inlet and outlet of a heat exchanger or of an evaporator (“boiler”) or into the inlet and outlet of a condenser. In addition, in the invention, the polymerization inhibitors may also be added to the olefinically unsaturated monomers in storage tanks.

In the context of this invention, the term “effective amount of the inventive polymerization inhibitor” is understood to mean the amount of polymerization inhibitor that is needed to prevent the premature polymerization of the olefinically unsaturated monomers. This effective amount of the polymerization inhibitor is dependent upon the conditions under which the olefinically unsaturated monomer is stored or handled. For example, in the case of distillation of the unsaturated monomer, a relatively high amount of the polymerization inhibitor is needed due to the relatively high temperatures and the relatively high concentration of impurities.

In the invention, from 100 ppb (m/m) to 10 000 ppm (m/m), more preferably from 1 ppm (m/m) to 1000 ppm (m/m) and most preferably from 10 ppm (m/m) to 100 ppm (m/m) of polymerization inhibitor is preferably added to the olefinically unsaturated monomer or to the monomer mixture, based on the olefinically unsaturated monomer.

In a preferred embodiment the inventive monomer composition includes a polymerization inhibitor having

from 0 to 100% by weight of a compound of the formula (1)

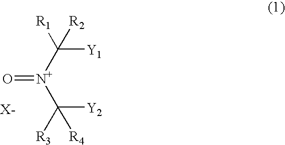

and

from 100 to 0% by weight of a compound of the formula (2)

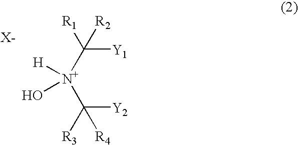

wherein
R₁, R₂, R₃ and R₄=alkyl group having from 1 to 4 carbon atoms,
Y₁, Y₂=alkyl group having from 1 to 4 carbon atoms, or Y₁ and Y₂ together form a ring system, and
X—=inorganic or organic anion,
   where the substituents of the R₁, R₂, R₃, R₄, Y₁ and Y₂ type are the same or different, either the substituents of the Y₁ and Y₂ type or the ring system based on the substituents of the Y₁ and Y₂ type may be unsubstituted or substituted, and the sum of the percentages by weight of the compounds of the formula (1) and (2) adds up to 100% by weight,

and contains at least one olefinically unsaturated monomer.

The inventive monomer composition may include either compounds of the formula (1) or of the formula (2) as polymerization inhibitors. The inventive monomer composition preferably contains both compounds of the formula (1) and of the formula (2) as polymerization inhibitors. In particular, the inventive monomer composition includes a polymerization inhibitor having

from 40 to 60% by weight of a compound of the formula (1) and

from 60 to 40% by weight of a compound of the formula (2).

The monomer composition more preferably contains a polymerization inhibitor having

from 45 to 55% by weight of a compound of the formula (1) and

from 55 to 45% by weight of a compound of the formula (2).

In a particular embodiment of the inventive monomer composition, the substituents of the Y₁ and Y₂ type in the compounds of the formulae (1) and (2) of the polymerization inhibitor are substituted, the substituent or these substituents of the substituents of the Y₁ and Y₂ type being selected from alkyl, ester, ether, hydroxyl, oxo, cyano, cyanohydrin, amino, amide, carboxyl, halogen, hydantoin, ketal, acetal or urethane group.

The two substituents of the Y₁ and Y₂ type of the compounds of the formulae (1) and (2) of the polymerization inhibitor together preferably form a ring system. More preferably, the inventive monomer composition includes a polymerization inhibitor having

from 0 to 100% by weight of a compound of the formula (3)

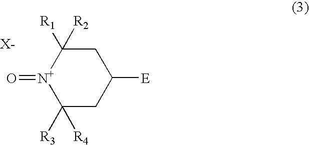

and

from 100 to 0% by weight of a compound of the formula (4)

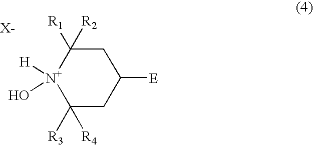

wherein
R₁, R₂, R₃ and R₄=alkyl group having from 1 to 4 carbon atoms,
E=alkyl, ester, ether, hydroxyl, oxo, cyano, cyanohydrin, amino, amide, carboxyl, halogen, hydantoin, ketal, acetal or urethane group and
X⁻=inorganic or organic anion,
   where the substituents of the R₁, R₂, R₃ and R₄ type are the same or different. Most preferably, the inventive monomer composition includes compounds of the formula (3) and/or (4) where R₁, R₂, R₃ and R₄=methyl group as polymerization inhibitor.

In particular, the inventive monomer composition has an inorganic or an organic anion in the compounds of the formulae (1) to (4); the composition preferably has inorganic anions. The inventive monomer composition more preferably has, as the anion X⁻ in the compounds of the formulae (1) to (4), a halogen anion, preferably a chlorine or a bromine anion.

The inventive monomer composition preferably has at least one olefinically unsaturated monomer selected from alk-1-enes or alka-1,3-dienes which may be either substituted or unsubstituted. The composition preferably contains olefinically unsaturated monomers selected from ethene, propene or propylene, butadiene, vinyl acetate, (meth)acrylic acid, (meth)acrylate, acrylonitrile, acrolein, N-vinylformamide, chloroprene, isoprene, divinylbenzene or styrene.

The inventive monomer composition may contain either one olefinically unsaturated monomer or a mixture of different olefinically unsaturated monomers.

The inventive monomer compositions preferably have from 100 ppb (m/m) to 10 000 ppm (m/m), more preferably from 1 ppm (m/m) to 1000 ppm (m/m) and most preferably from 10 ppm (m/m) to 100 ppm (m/m) of polymerization inhibitor, based on the olefinically unsaturated monomer.

The examples that follow are intended to illustrate the invention in detail, with no intention that the invention be restricted to these embodiments.

Preparation of the polymerization inhibitors:

### EXAMPLE 1

In Example 1, no polymerization inhibitor was used.

### EXAMPLE 2

In Example 2, the polymerization inhibitor used was commercially available 4-hydroxy-TEMPO from Degussa without further workup.

### EXAMPLE 3

In Example 3, the polymerization inhibitor was prepared by a process that is described by Paleos et al. in “Ready Reduction of Some Nitroxide Free Radicals Using Ascorbic Acid” (J. Chem. Soc., Chem. Comm., 1997, 345-346).

### EXAMPLES 4 AND 5

In Examples 4 and 5, the polymerization inhibitors were prepared by a process that is described by Merbouh et al. in the experimental part on pages 24 to 26 in “Preparation of tetramethylpiperidine-1-oxoammonium salts and their use as oxidants in organic chemistry. A review” (Organic Preparations and Procedures International, 2004, 36(1), 3-31) or Golubev et al. in “Some Reactions Of Free Iminoxyl Radicals With The Participation Of The Unpaired Electron” (Izvestiya Akademii Nauk SSSR, Seriya Khimicheskaya, No. 11, 1965, 1927-1936). The reactant used was the 4-hydroxy-TEMPO from Degussa. It was demonstrated analytically that the polymerization inhibitor of Example 5 was the monobromide.

### EXAMPLE 6

The polymerization inhibitor used in Example 6 was prepared by dissolving 1 g of 1,4-dihydroxy-2,2,6,6-tetramethylpiperidine in 200 ml of degassed dichloroethane and then passing HCl gas into the solution at room temperature slowly for one hour. After postreaction for one hour, the precipitated yellow solid was filtered off, washed with tertbutyl methyl ether and dried.

### EXAMPLES 7 AND 8

The polymerization inhibitor used in Examples 7 and 8 was prepared by dissolving 0.05 mol of 4-hydroxy-TEMPO from Degussa in 200 g of dichloroethane and then introducing 0.1 mol of HCl gas into the solution slowly with gentle cooling at room temperature for one hour. After one hour of postreaction, the precipitated yellow solid was filtered off, washed with dichloromethane and dried.

### Experiments on the Stability of the Monomer Mixture:

Commercially available styrene (from Fluka, purum, monomer, ≧99.0% (GC)) is freed of the tert-butyl-1,2-hydroxybenzene stabilizer at a reduced pressure of 95 hPa, a bottom temperature of 75° C. and a nitrogen blanket. A three-neck flask with a thermometer, condenser, septum and a magnetic stirrer is purged thoroughly with nitrogen in order to obtain an oxygen-free atmosphere in the flask. This nitrogen atmosphere is retained throughout the duration of the experiment. The three-neck flask is charged with precisely 300 g of the distilled styrene and a particular amount of polymerization inhibitor. In order to start the experiment, the three-neck flask is immersed into an oil bath preheated to 110° C., in the course of which the stabilized monomer composition present in the three-neck flask is stirred. The amount and the type of the polymerization inhibitor is shown in Table 1. After immersion of the three-neck flask into the heated oil bath, 2 g of the monomer composition are taken as a sample at regular intervals, weighed accurately and introduced into 10 ml of methanol. After 30 minutes, the polystyrene precipitated from methanol is removed by means of a glass filter crucible, dried at 110° C. for 5 hours and weighed accurately. The amount of polystyrene present in the sample of the monomer composition is plotted against the reaction time in a diagram. As a comparative value between the examples, the time at which the sample of the monomer composition has attained a polymer content of 3% by weight according to the measurement is determined or interpolated if necessary for each polymerization inhibitor.

TABLE 1

| | | | Time after which the |
|---|---|---|---|
| | | | sample of the monomer |
| | | Active amount of | composition had |
| | | polymerization | attained a polymer |
| | | inhibitor | content of 3% by weight |
| Example | Polymerization inhibitor | [in ppm (m/m)] | [in minutes] |
| 1 | — | — | 50 |
| 2 | 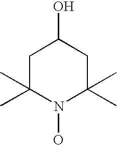 | 100 | 120 |
| 3 | 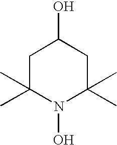 | 100 | 90 |
| 4 | 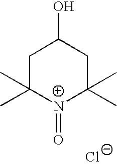 | 100 *) | 145 |
| 5 | 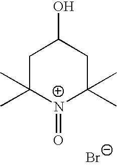 | 100 *) | 210 |
| 6 | 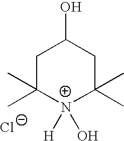 | 100 *) | 255 |
| 7 | 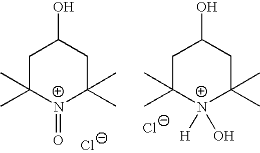 | 50 each (100 intotal) *) | 210 |
| 8 | 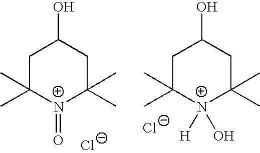 | 50 each (100 intotal) **) | 205 |

*) In the calculation of the amount of polymerization inhibitors, only the molecular weight of the cation and not of the anion was taken into account for the ionic compounds.
**) In Example 8, in contrast to Example 7, the molecular weight of the anion was also taken into account.

Examples 4-8 are examples of the present invention.

The above written description of the invention provides a manner and process of making and using it such that any person skilled in this art is enabled to make and use the same, this enablement being provided in particular for the subject matter of the appended claims, which make up a part of the original description and including a method for stabilizing an olefinically unsaturated monomer, comprising mixing at least one olefinically unsaturated monomer with a polymerization inhibitor composition comprising at least one compound of formulae (1) and/or (2):

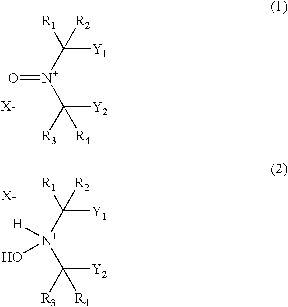

where:
R₁, R₂, R₃ and R₄=alkyl group having from 1 to 4 carbon atoms,
Y₁, Y₂=alkyl group having from 1 to 4 carbon atoms, or Y₁ and Y₂ together form a ring system, and
X⁻=inorganic or organic anion,
   where the substituents R₁, R₂, R₃, R₄, Y₁ and Y₂ are the same or different, and where Y₁ and Y₂ and the ring system based on Y₁ and Y₂ may be unsubstituted or substituted, and the resultant stabilized compositions. As used herein, a polymerization inhibitor composition comprising at least one compound of formulae (1) and/or (2) describes compositions comprising a) one or more compounds of formula (1), b) one or more compounds of formula (2), and c) one or more compounds of formula (1) and one or more compounds of formula (2).

As used herein, the phrases “selected from the group consisting of,” “chosen from,” and the like include mixtures of the specified materials. Terms such as “contain(s),” “includes” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for stabilizing an olefinically unsaturated monomer, comprising mixing at least one olefinically unsaturated monomer and a polymerization inhibitor composition comprising at least one compound of formulae (1) and/or (2): 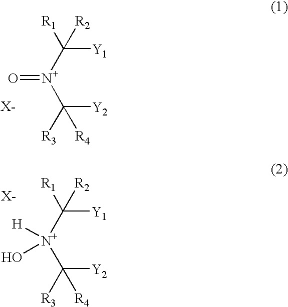
where:
R₁, R₂, R₃ and R₄=alkyl group having from 1 to 4 carbon atoms,
Y₁, Y₂=alkyl group having from 1 to 4 carbon atoms, or Y₁ and Y₂ together form a ring system, and
X⁻=inorganic or organic anion,
where the substituents R₁, R₂, R₃, R₄, Y₁ and Y₂ are the same or different, and where Y₁ and Y₂ and the ring system based on Y₁ and Y₂ may be unsubstituted or substituted.

2. The method to claim 1, wherein Y₁ and Y₂ are substituted with one or more substituents selected from alkyl, ester, ether, hydroxyl, oxo, cyano, cyanohydrin, amino, amide, carboxyl, halogen, hydantoin, ketal, acetal and urethane group.

3. The method according to claim 1,
wherein the at least one olefinically unsaturated monomer is selected from the group consisting of ethene, propene, propylene, butadiene, vinyl acetate, (meth)acrylic acid, (meth)acrylate, acrylonitrile, acrolein, N-vinyl-formamide, chloroprene, isoprene, divinylbenzene, styrene, and mixtures thereof.

4. The method according to claim 1,
wherein
from 100 ppb (m/m) to 10 000 ppm (m/m) of at least one compound of formula (1) and/or (2) is added to the monomer, based on the olefinically unsaturated monomer.

5. The method according to claim 1,
wherein
from 1 ppm (m/m) to 1000 ppm (m/m) of at least one compound of formula (1) and/or (2) is added to the monomer, based on the olefinically unsaturated monomer.

6. The method according to claim 1,
wherein the compound of formula (1) is a compound of the formula (3) 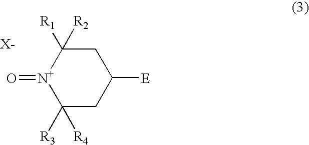
and
the compound of formula (2) is a compound of the formula (4) 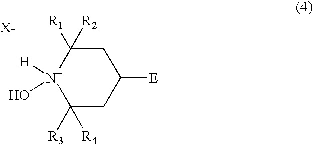
wherein
R₁, R₂, R₃ and R₄=alkyl group having from 1 to 4 carbon atoms,
E=alkyl, ester, ether, hydroxyl, oxo, cyano, cyanohydrin, amino, amide, carboxyl, halogen, hydantoin, ketal, acetal or urethane group and
X⁻=inorganic or organic anion,
wherein the substituents of the R₁, R₂, R₃ and R₄ type are the same or different.

7. A monomer composition, comprising
at least one olefinically unsaturated monomer and a polymerization inhibitor composition comprising at least one compound of formulae (1) and/or (2) 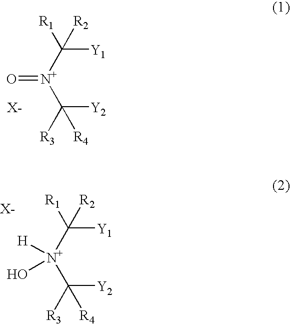
wherein
R₁, R₂, R₃ and R₄=alkyl group having from 1 to 4 carbon atoms and
Y₁, Y₂=alkyl group having from 1 to 4 carbon atoms, or
Y₁ and Y₂=together form a ring system, and
X⁻=inorganic or organic anion,
where R₁, R₂, R₃, R₄, Y₁ and Y₂ are the same or different, and Y₁ and Y₂ and the ring system based on Y₁ and Y₂ type may be unsubstituted or substituted.

8. The method according to claim 1,
wherein in the polymerization inhibitor composition the compound of formula (1) is present in an amount of 40 to 60% by weight and the compound of formula (2) is present in an amount of 60 to 40% by weight relative to the total weight of compounds of formulae (1) and (2).

9. The method according to claim 8,
wherein in the polymerization inhibitor composition the compound of formula (1) is present in an amount of 45 to 55% by weight and the compound of formula (2) is present in an amount of 55 to 45% by weight relative to the total weight of compounds of formulae (1) and (2).

10. The method according to claim 1, wherein the polymerization inhibitor comprises at least one compound of formula (1).

11. The method according to claim 1, wherein the polymerization inhibitor comprises at least one compound of formula (2).

12. The monomer composition according to claim 7,
wherein in the polymerization inhibitor composition the compound of formula (1) is present in an amount of 40 to 60% by weight and the compound of formula (2) is present in an amount of 60 to 40% by weight relative to the total weight of compounds of formulae (1) and (2).

13. The monomer composition according to claim 12,
wherein in the polymerization inhibitor composition the compound of formula (1) is present in an amount of 45 to 55% by weight and the compound of formula (2) is present in an amount of 55 to 45% by weight relative to the total weight of compounds of formulae (1) and (2).

14. The monomer composition according to claim 7, wherein the polymerization inhibitor composition comprises at least one compound of formula (1).

15. The monomer composition according to claim 7, wherein the polymerization inhibitor composition comprises at least one compound of formula (2).

16. The monomer composition according to claim 7, wherein the compound of formula (1) is a compound of the formula (3) 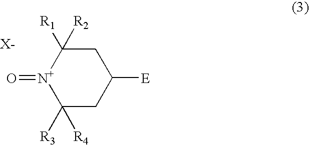
and
the compound of formula (2) is a compound of the formula (4) 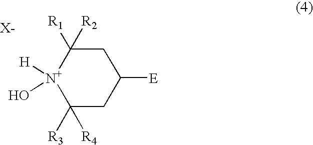
wherein
R₁, R₂, R₃ and =alkyl group having from 1 to 4 carbon atoms,
E=alkyl, ester, ether, hydroxyl, oxo, cyano, cyanohydrin, amino, amide, carboxyl, halogen, hydantoin, ketal, acetal or urethane group and
X⁻=inorganic or organic anion,
wherein the substituents of the R₁, R₂, R₃ and R₄ type are the same or different.

17. The monomer composition according to claim 16, wherein the polymerization inhibitor composition comprises at least one compound of formula (3).

18. The monomer composition according to claim 16, wherein the polymerization inhibitor composition comprises at least one compound of formula (4).
